(19) United States
(12) Patent Application Publication
(10) Pub. No.: US 2008/0300500 A1
(43) Pub. Date: Dec. 4, 2008

(54) APNEA DETECTION USING A CAPNOGRAPH

(75) Inventor: Daniel Reisfeld, Tel Aviv (IL)

Correspondence Address: DARBY & DARBY P.C., P.O. BOX 770, Church Street Station, New York, NY, 10008-0770, US

(73) Assignee: WIDEMED LTD., Herzlia (IL)

(21) Appl. No.: 11/755,568

(22) Filed: May 30, 2007

(51) Int. Cl. A61B 5/08

(52) U.S. Cl. 600/532

(57) ABSTRACT

A method for diagnosis includes receiving a signal indicative of a partial pressure of CO₂ in air expired by a patient during sleep. The signal is processed so as to detect a breathing-related event from a group of events consisting of apneas and hypopneas, and to classify the event as a central event or an obstructive event.

### FIELD OF THE INVENTION

The present invention relates generally to physiological monitoring and diagnosis, and specifically to detection and classification of breathing disorders.

### BACKGROUND OF THE INVENTION

Sleep apnea is commonly defined as a cessation of airflow for more than 10 sec. This term is distinguished from “hypopnea,” which is a reduction, but not complete cessation, of airflow to less than 50% of normal (usually in association with a reduction in oxyhemoglobin saturation).

Sleep apneas and hypopneas are generally believed to fall into two categories: obstructive, due to collapse of the pharynx; and central, due to withdrawal of central respiratory drive to the muscles of respiration. Central sleep apnea (CSA) is commonly associated with Cheyne-Stokes respiration, which is a form of periodic breathing in which central apneas and hypopneas alternate with periods of hyperventilation, with a waxing-waning pattern of tidal volume. CSA is believed to arise as the result of heart failure, though obstructive sleep apnea (OSA) may also occur in heart failure patients. Detailed criteria for classification of apneas and hypopneas are presented by an American Academy of Sleep Medicine Task Force, in “Sleep-Related Breathing Disorders in Adults: Recommendations for Syndrome Definition and Measurement Techniques in Clinical Research,” SLEEP 22:5 (1999), pages 667-669, which is incorporated herein by reference.

Various methods have been proposed in the patent literature for automated apnea detection and diagnosis based on patient monitoring during sleep. For example, U.S. Patent Application Publication US 2004/0230105 A1 describes a method for analyzing respiratory signals using a Fuzzy Logic Decision Algorithm (FLDA). The method may be used to associate respiratory disorders with obstructive apnea, hypopnear central apnea, or other conditions. As another example, PCT International Publication WO 2006/082589 describes a method for patient monitoring in which a respiration-related signal is processed to detect periodic breathing patterns during sleep. A shape characteristic of the periodic breathing pattern, such as the symmetry of the pattern, is used in classifying the etiology of the episode, by determining the episode to be predominantly obstructive or central in origin. Other methods for apnea diagnosis are described in U.S. Patent Application Publication US 2002/0002327 A1, U.S. Pat. No. 6,839,581, U.S. Pat. No. 6,760,608 and U.S. Pat. No. 6,856,829. The disclosures of the patents and publications cited above are incorporated herein by reference.

### SUMMARY OF THE INVENTION

Embodiments of the present invention provide methods and systems for diagnosis of patient conditions based on analysis of patient breathing. In some of these embodiments, a capnograph measures partial pressure of CO₂ in the air expired by the patient. Characteristics of the capnograph waveform are analyzed in order to detect and classify apneas and hypopneas. The capnograph may be used on its own for this purpose or in conjunction with other patient monitoring devices.

There is therefore provided, in accordance with an embodiment of the present invention, a method for diagnosis, including:

receiving a signal indicative of a partial pressure of CO₂ in air expired by a patient during sleep;

processing the signal so as to detect a breathing-related event from a group of events consisting of apneas and hypopneas, and to classify the event as a central event or an obstructive event; and

generating a record of an occurrence and classification of the event.

Typically, processing the signal includes detecting a repetitive waveform in the signal prior to the event, and comparing the signal during the event to the detected waveform. In some embodiments, comparing the signal includes detecting an apnea responsively to an interruption of the repetitive waveform. Processing the signal may include classifying the apnea as central or obstructive responsively to a level of the signal during the interruption, wherein detecting the repetitive waveform includes finding peak and baseline values of the repetitive waveform, and wherein classifying the apnea includes identifying a central apnea when the level is closer to the peak value than to the baseline value, and identifying an obstructive apneas when the level is closer to the baseline value than to the peak value.

In some embodiments, detecting the repetitive waveform includes finding shape parameters of the waveform, and comparing the signal includes detecting a hypopnea responsively to a change in one or more of the shape parameters. The shape parameters may include a respective slope and a respective duration of one or more phases of the waveform, and processing the signal may include classifying the hypopnea as obstructive responsively to a change in the respective slope and the respective duration of at least one of the phases.

In another embodiment, detecting the repetitive waveform includes finding a peak level of the waveform, and comparing the signal includes detecting a hypopnea responsively to an increase in the peak level over multiple cycles of the repetitive waveform. Processing the signal may include identifying a pattern of Cheyne-Stokes breathing responsively to a succession of alternating increases and decreases of the peak level.

In a disclosed embodiment, generating the record includes detecting and recording an occurrence of Cheyne-Stokes breathing, and the method includes determining a prognosis of heart failure (HF) in the patient based on the occurrence of the Cheyne-Stokes breathing.

There is also provided, in accordance with an embodiment of the present invention, apparatus for diagnosis, including:

a sensor, which is configured to be coupled to a body of a patient during sleep and to output a signal indicative of a partial pressure of CO₂ in air expired by a patient; and

a processor, which is coupled to process the signal so as to detect a breathing-related event from a group of events consisting of apneas and hypopneas, and to classify the event as a central event or an obstructive event.

There is additionally provided, in accordance with an embodiment of the present invention, a computer-software product, including a computer-readable medium in which program instructions are stored, which instructions, when read by a computer, cause the computer to receive a signal indicative of a partial pressure of CO₂ in air expired by a patient during sleep, and to process the signal so as to detect a breathing-related event from a group of events consisting of apneas and hypopneas, and to classify the event as a central event or an obstructive event.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a system for patient monitoring during sleep, in accordance with an embodiment of the present invention;

FIGS. 2A and 2B are schematic plots of physiological signals received from patients during sleep, in accordance with an embodiment of the present invention;

FIG. 3 is a schematic plot of a capnograph waveform, which is analyzed in accordance with an embodiment of the present invention;

FIGS. 4A and 4B are schematic plots of capnograph signals received from patients during sleep, in accordance with an embodiment of the present invention; and

FIG. 5 is a schematic plot of airflow and capnograph signals received from a patient during sleep, in accordance with an embodiment of the present invention.

### DETAILED DESCRIPTION OF EMBODIMENTS

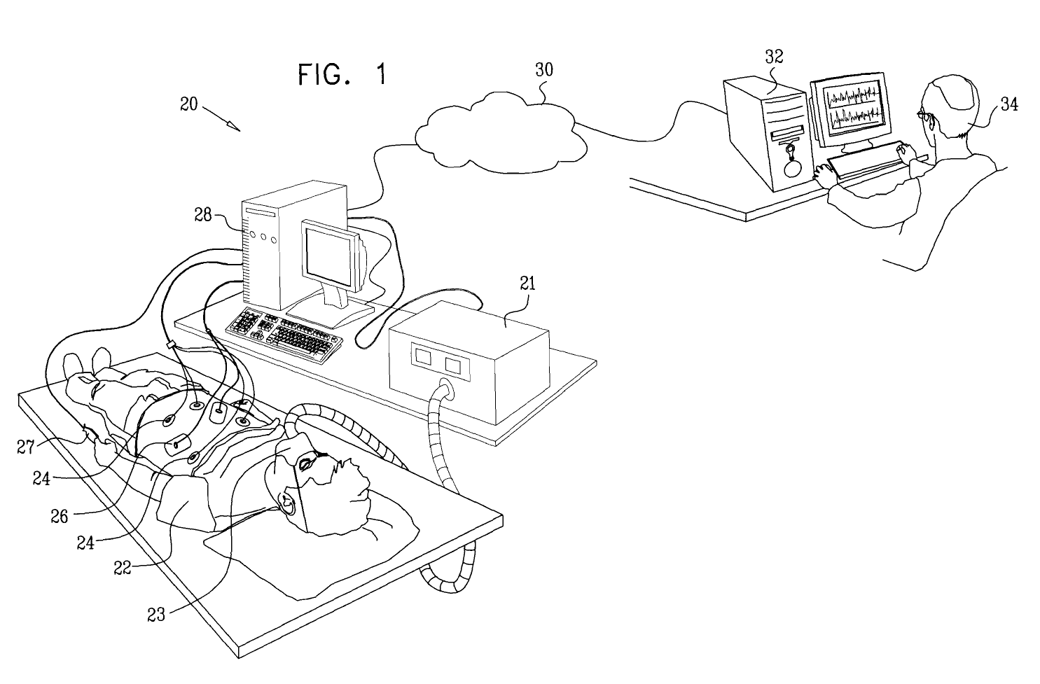

FIG. 1 is a schematic, pictorial illustration of a system 20 for sleep monitoring and diagnosis, in accordance with an embodiment of the present invention. In this embodiment, system 20 is used to monitor a patient 22 in a home, clinic or hospital ward environment, although the principles of the present invention may similarly be applied in dedicated sleep laboratories. System 20 receives and analyzes a signal from a capnograph 21, which serves as a sensor to determine the partial pressure of CO₂ (PaCO2) in air exhaled by patient 22. The expired air may be captured, for example, by a mask 23 or other suitable device.

Optionally, system 20 may also receive other physiological signals generated by the patient's body, such as an ECG signal measured by skin electrodes 24 and/or a respiration signal measured by a respiration sensor 26. Additionally or alternatively, the system may comprise a photoplethysmograph device 27, which serves as an oxygen saturation sensor. The sensor signals are collected, amplified and digitized by a console 28. Although no EEG or EOG electrodes are shown in FIG. 1, the techniques of monitoring and analysis that are described herein may alternatively be combined with EEG, EOG, leg motion sensors, and other sleep monitoring modalities that are known in the art.

Respiration sensor 26 typically makes electrical measurements of thoracic and abdominal movement. For example, sensor 26 may comprise two or more skin electrodes, which are driven by console 28 to make a plethysmographic measurement of the change in impedance or inductance between the electrodes as a result of the patient's respiratory motion. (It is also possible to use the ECG electrodes for this purpose.) Alternatively or additionally, the respiration sensor may comprise a belt, which is placed around the patient's chest or abdomen and senses changes in the body perimeter. Further additionally or alternatively, measurement of flow through the patient's airway may be used for respiration sensing. For example, the airflow from the patient's nose and/or mouth may be measured using a pressure cannula or thermistor associated with mask 23, or by capnograph 21. Any other suitable respiration sensor known in the art may also be used, in addition to or instead of the above sensor types.

Console 28 may process and analyze the ECG, respiration and other signals locally, using the methods described hereinbelow. In the present embodiment, however, console 28 is coupled to communicate over a network 30, such as a telephone network or the Internet, with a diagnostic processor 32. This configuration permits sleep studies to be performed simultaneously in multiple different locations. Processor 32 typically comprises a general-purpose computer with suitable software for carrying out the functions described herein. This software may be downloaded to processor 32 in electronic form, or it may alternatively be provided on tangible media, such as optical, magnetic or non-volatile electronic memory. Processor 32 analyzes the signals conveyed by console 28 in order to identify and classify breathing-related events, and to generate a record and analysis of these events in a memory of the processor and/or on an output device, such as a display read by an operator 34.

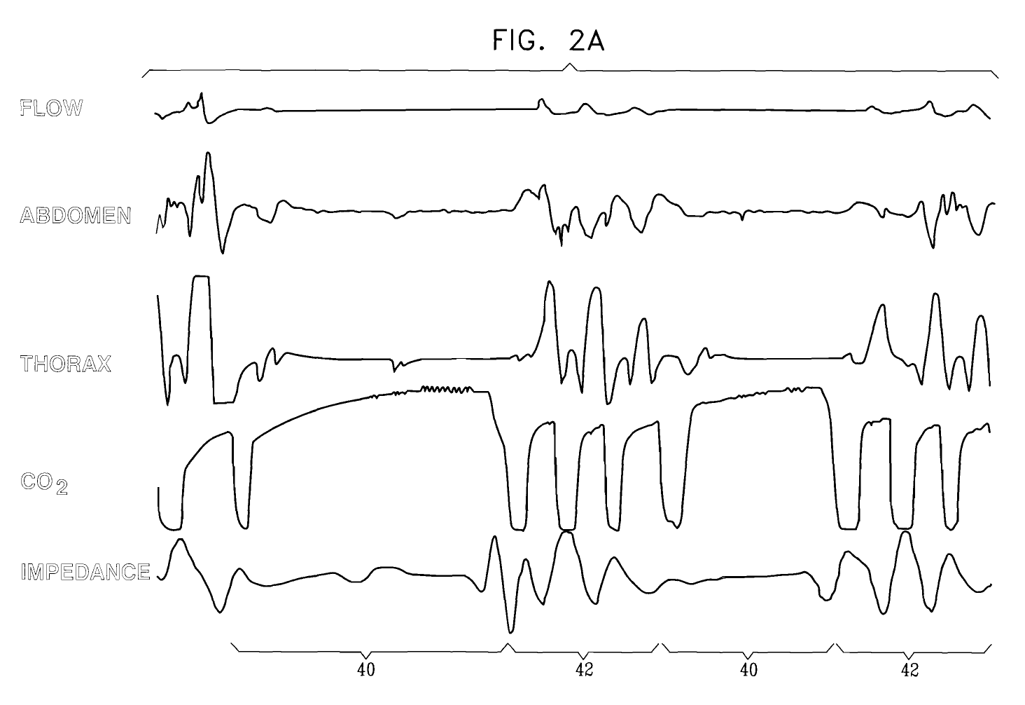

FIGS. 2A and 2B are schematic plots of respiration-related physiological signals received by system 20, in accordance with an embodiment of the present invention. The signals, as explained above, include measurements of airflow, abdominal and thoracic movement, PaCO2, and plethysmographic impedance. FIG. 2A shows two episodes of central apnea 40 followed by hyperpneas 42 (increased breathing effort), while FIG. 2B shows two episodes of obstructive apnea 44 followed by hyperpneas 46.

Episodes 40 and 44 are similar in that they are characterized by cessation of the repetitive waveforms generated by breathing activity, as reflected in the measurements of airflow, movement and impedance. The two types of apnea differ markedly, however, in the PaCO2 characteristic:
During central apneas 40, the patient's lungs continue to absorb oxygen from the inhaled air and to produce CO₂. Because the patient's airway is open, the CO₂ from the lungs reaches mask 23, and the partial pressure of CO₂ that is measured by capnograph 21 is high, at a level close to or greater than the peak of the PaCO2 waveform prior to the apnea.
During obstructive apneas 44, the patient's airway is closed, and the CO₂ in the lungs cannot reach mask 23. Therefore, the partial pressure of CO₂ measured by the capnograph remains low, near the baseline of the pre-apnea waveform.

In both cases, the PaCO2 signal remains at the respective level (high or low) for an extended period, as the periodic waveform that characterized the normal breathing rhythm is interrupted.

Thus, processor 32 may use the PaCO2 measurement (by itself or in conjunction with other signals) in order to detect episodes of apnea and to classify the episodes as central or obstructive, depending on the PaCO2 level. When the repetitive waveform of normal breathing is interrupted for a certain minimal amount of time, a PaCO2 level closer to the peak than to the baseline of the waveform indicates a central apnea, whereas a PaCO2 level closer to the baseline than to the peak indicates an obstructive apnea. Mixed apnea episodes, which typically begin as a central apnea followed immediately by obstructive apnea, may be manifested in a transition from the high PaCO2 level of the apneas in FIG. 2A to the low PaCO2 level of those in FIG. 2B, without immediate resumption of the normal breathing rhythm.

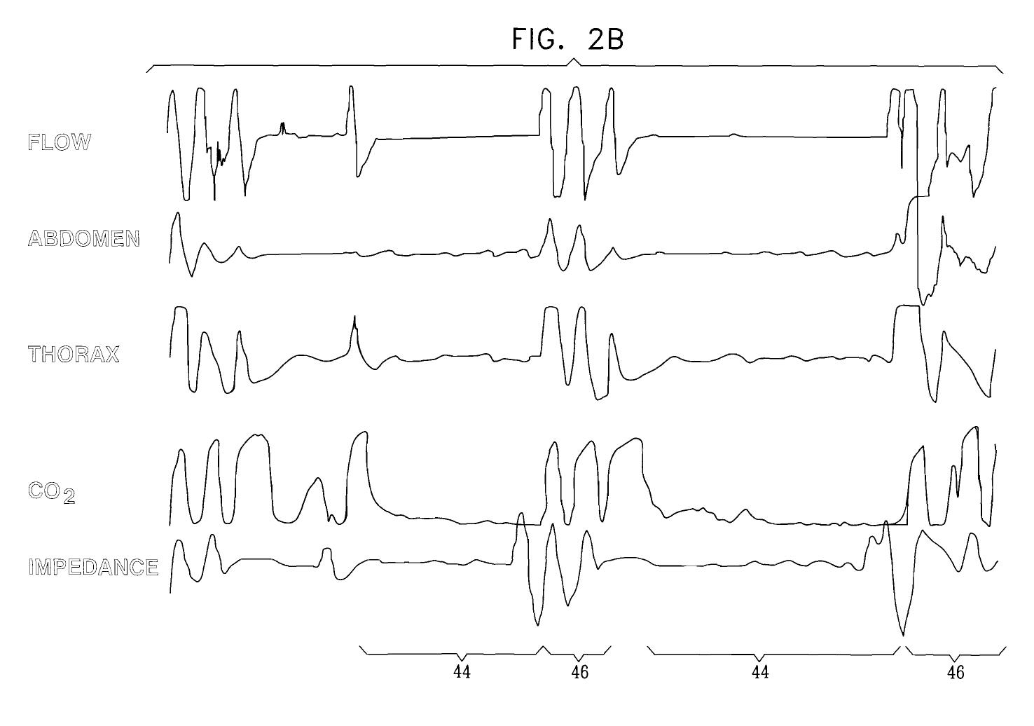

FIG. 3 is a schematic plot of a capnograph waveform 50, which is analyzed by processor 32 in accordance with an embodiment of the present invention. This analysis is useful particularly in classifying hypopneas, as is explained further hereinbelow. Waveform 50 comprises four phases:
Phase 0—inhalation.
Phase I—initial exhalation. During this phase, the gas received by the capnograph comes from anatomical dead space, in which there is no exchange of O₂ for CO₂, and PaCO2 is therefore low.
Phase II—peak exhalation. In this phase, CO₂-rich gas from the alveoli mixes with dead-space gas, giving a rapid increase in PaCO2.
Phase III—final tide of exhalation (alveolar plateau). The gas reaching the capnograph in this phase is entirely from the alveoli, but PaCO2 continues to rise as the CO₂ concentration in the blood continues to increase until inhalation begins again.

To analyze waveform 50, processor 32 fits a piecewise-linear function 52 to the waveform. For this purpose, low and high thresholds, T₁ and T₂, are defined. T₁ may be set, for example, to 30% of the median end-tidal PaCO2 value (the value at the end of phase III, which is typically about 40 mm Hg), while T₂ is set to 70% of the mean end-tidal value. The processor computes shape parameters, including the durations and slopes of certain phases of the signal. For example, the processor may find the duration of phase II, d_{II}, by taking the time elapsed between the points at which waveform 50 passes through the thresholds T₁ and T₂. The slope of phase II, θ_{II}, is computed by linear regression through the sampling points in waveform 50 between T₁ and T₂. The duration and slope of phase III, d_{III} and θ_{III}, are computed in like manner, except that T₂ and the end-tidal PaCO2 values are taken as the bounding thresholds. Optionally, processor 32 may also compute the area under waveform 50, as well as the width of the waveform, which is given by the time difference between the points at which the rising and falling edges of the waveform pass a certain value, such as T₂.

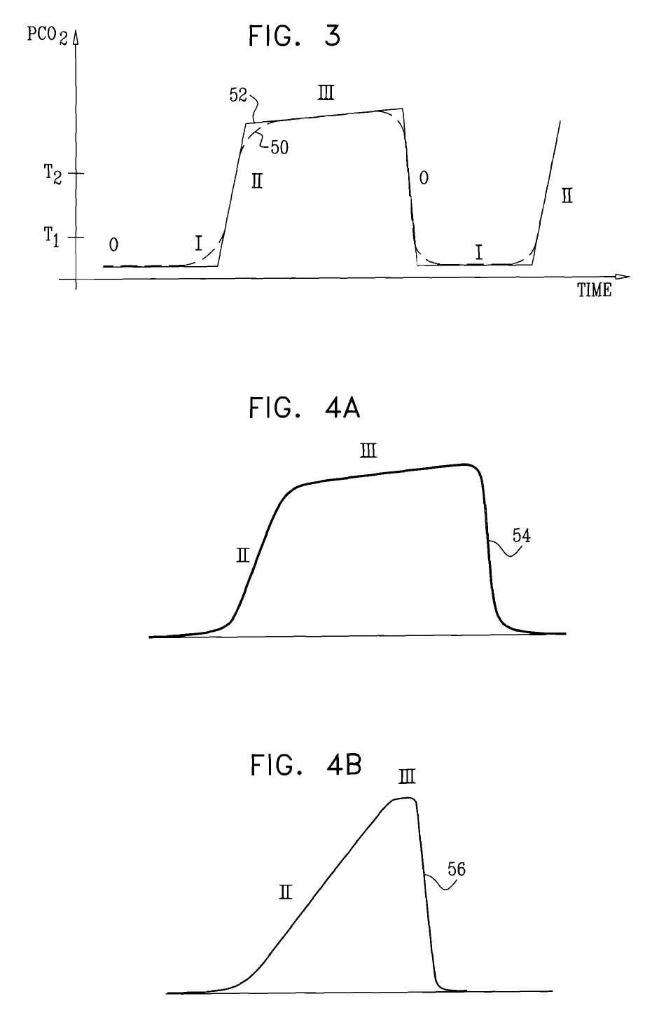

FIGS. 4A and 4B are schematic plots of capnograph waveforms 54 and 56, respectively, based on signals received from patients during sleep, in accordance with an embodiment of the present invention. Waveform 54 corresponds to a normal breath, while waveform 56 was captured during an episode of obstructive hypopnea. Partial occlusion of the airway during the hypopnea slows the release of CO₂ from the lungs, resulting in a longer d_{II} and shorter d_{III}, as well as a smaller slope θ_{II} and possibly a higher end-tidal PaCO2 value. During an episode of hypopnea, waveform 56 will recur over multiple, successive breaths, with increasing end-tidal PaCO2 from breath to breath.

Thus, in order to detect obstructive hypopnea events, processor 32 monitors variations in the slope 0_{II} and in the relative lengths of d_{II} and d_{III} over multiple breaths. A reduction in slope and increase in d_{II}/d_{III} ratio is indicative of an obstructive hypopnea. Additionally or alternatively, the processor may use the width and/or area under the capnograph waveform for this purpose. Increasing end-tidal PaCO2 over a sequence of breaths with low slope and high d_{II}/d_{III} ratio is an added sign of the hypopnea. Optionally, the processor may also check the patient's blood oxygen saturation, since hypopneas are generally characterized by a decrease of at least 3-4% in the saturation level.

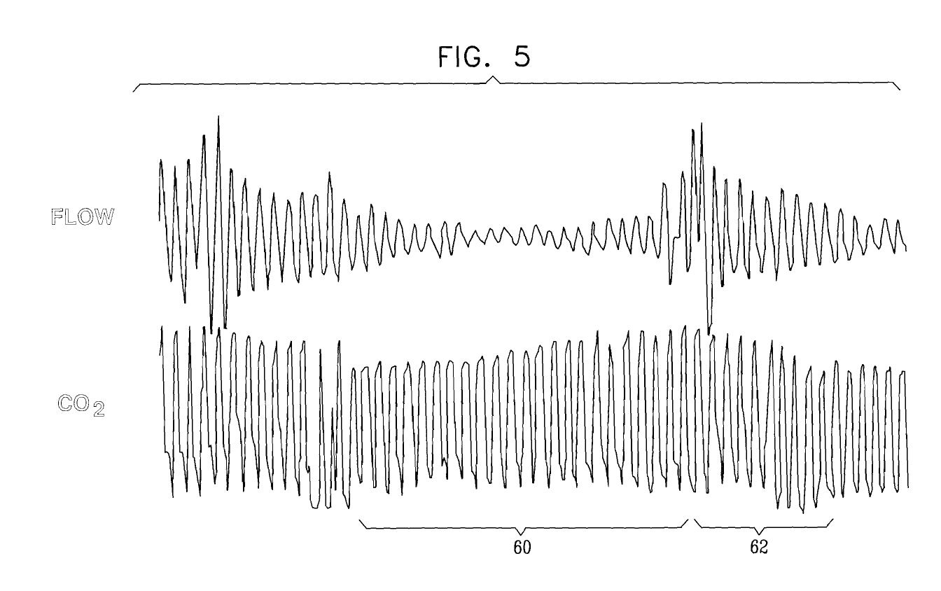

FIG. 5 is a schematic plot of airflow and capnograph signals received from a patient during sleep, in accordance with another embodiment of the present invention. In this case, an episode of central hypopnea 60 is marked by reduced flow, without a significant change in the shape of the capnograph waveform (in contrast to the shape changes shown above for obstructive hypopnea). At each successive breath during hypopnea 60, however, the end-tidal PaCO2 level increases, due to the patient's decreased breath volume, notwithstanding the unobstructed airway. The end-tidal PaCO2 level then drops off during a hyperpnea 62 following the hypopnea.

Therefore, if processor 32 detects a substantial increase in end-tidal PaCO2 over multiple breaths, without significant changes in the shape of the capnograph waveform, it may conclude that patient 22 has undergone an episode of central hypopnea. To determine whether the increase in the end-tidal PaCO2 is significant, the processor may fit a line through the end-tidal values. The processor records a central hypopnea event if the end-tidal value increases on average by greater than 10% from breath to breath. The processor may confirm the finding of central hypopnea by checking for a drop of at least 3-4% in the blood oxygen saturation level. During hyperpnea 62, the slope of the end-tidal PaCO2 values will turn negative, hence providing an additional indication that a hypopnea has occurred.

Thus, based on the principles exemplified in the preceding figures, processor 32 is able to detect and differentiate between central and obstructive apneas and hypopneas, by processing the PaCO2 signal provided by capnograph 21. Optionally, though not necessarily, the processor may use other physiological signals, such as readings of blood oxygen saturation, respiratory motion and air flow, to supplement PaCO2 readings and confirm the occurrence and classification of apneas and hypopneas.

Processor 32 may record and analyze the sequence of apnea and hypopnea events that are detected during a night's sleep in order to diagnose and determine the prognosis of diseases affecting patient 22. For this purpose, the processor may apply, for example, methods described in the above-mentioned PCT International Publication WO 2006/082589, as well as in U.S. patent application Ser. No. 11/750,222, filed May 17, 2007, which is assigned to the assignee of the present patent application and whose disclosure is incorporated herein by reference.

Although this latter patent application refers particularly to analysis of photoplethysmograph signals, the methods described in the application may similarly be applied, mutatis mutandis, to other types of respiration-related signals, such as capnograph signals. Thus, for example, processor 32 may filter the PaCO2 signal to extract low-frequency components, typically in the range of 1/90- 1/45 Hz. When patients suffering from cyclic breathing disorders, such as Cheyne-Stokes breathing, are monitored, the maxima in the filtered PaCO2 signal will occur at the end of each apnea or hypopnea, while minima will occur at the end of the subsequent hyperpnea. For Cheyne-Stokes patients, the time span between successive maxima or between successive minima will be in the range of 1 minute (typically 45-90 sec). When the processor identifies a sequence of alternating maxima and minima of this sort that extend over a given minimum time, such as 10 minutes, with a difference between successive maxima and minima that is greater than a given threshold percentage, it marks the sequence as an episode of Cheyne-Stokes breathing. The processor may record and quantify such episodes in order to determine the prognosis and recommended treatment for heart failure patients, as described in the above-mentioned U.S. patent application.

It will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A method for diagnosis, comprising:
receiving a signal indicative of a partial pressure of CO₂ in air expired by a patient during sleep;
processing the signal so as to detect a breathing-related event from a group of events consisting of apneas and hypopneas, and to classify the event as a central event or an obstructive event; and
generating a record of an occurrence and classification of the event.

2. The method according to claim 1, wherein processing the signal comprises detecting a repetitive waveform in the signal prior to the event, and comparing the signal during the event to the detected waveform.

3. The method according to claim 2, wherein comparing the signal comprises detecting an apnea responsively to an interruption of the repetitive waveform.

4. The method according to claim 3, wherein processing the signal comprises classifying the apnea as central or obstructive responsively to a level of the signal during the interruption.

5. The method according to claim 4, wherein detecting the repetitive waveform comprises finding peak and baseline values of the repetitive waveform, and wherein classifying the apnea comprises identifying a central apnea when the level is closer to the peak value than to the baseline value, and identifying an obstructive apneas when the level is closer to the baseline value than to the peak value.

6. The method according to claim 2, wherein detecting the repetitive waveform comprises finding shape parameters of the waveform, and wherein comparing the signal comprises detecting a hypopnea responsively to a change in one or more of the shape parameters.

7. The method according to claim 6, wherein the shape parameters comprise a respective slope and a respective duration of one or more phases of the waveform, and wherein processing the signal comprises classifying the hypopnea as obstructive responsively to a change in the respective slope and the respective duration of at least one of the phases.

8. The method according to claim 2, wherein detecting the repetitive waveform comprises finding a peak level of the waveform/ and wherein comparing the signal comprises detecting a hypopnea responsively to an increase in the peak level over multiple cycles of the repetitive waveform.

9. The method according to claim 8, wherein processing the signal comprises identifying a pattern of Cheyne-Stokes breathing responsively to a succession of alternating increases and decreases of the peak level.

10. The method according to claim 1, wherein generating the record comprises detecting and recording an occurrence of Cheyne-Stokes breathing.

11. The method according to claim 10, and comprising determining a prognosis of heart failure (HF) in the patient based on the occurrence of the Cheyne-Stokes breathing.

12. Apparatus for diagnosis, comprising:
a sensor, which is configured to be coupled to a body of a patient during sleep and to output a signal indicative of a partial pressure of CO₂ in air expired by a patient; and
a processor, which is coupled to process the signal so as to detect a breathing-related event from a group of events consisting of apneas and hypopneas, and to classify the event as a central event or an obstructive event.

13. The apparatus according to claim 12, wherein the processor is configured to detect a repetitive waveform in the signal prior to the event, and to detect the event by comparing the signal during the event to the detected waveform.

14. The apparatus according to claim 13, wherein the processor is configured to detect an apnea responsively to an interruption of the repetitive waveform.

15. The apparatus according to claim 14, wherein the processor is configured to classify the apnea as central or obstructive responsively to a level of the signal during the interruption.

16. The apparatus according to claim 15, wherein the processor is configured to find peak and baseline values of the repetitive waveform, and to classify the apnea as a central apnea when the level is closer to the peak value than to the baseline value, and as an obstructive apneas when the level is closer to the baseline value than to the peak value.

17. The apparatus according to claim 13, wherein the processor is configured to find shape parameters of the waveform, and to detect a hypopnea responsively to a change in one or more of the shape parameters.

18. The apparatus according to claim 17, wherein the shape parameters comprise a respective slope and a respective duration of one or more phases of the waveform, and wherein the processor is configured to classify the hypopnea as obstructive responsively to a change in the respective slope and the respective duration of at least one of the phases.

19. The apparatus according to claim 13, wherein the processor is configured to find a peak level of the waveform, and to detect a hypopnea responsively to an increase in the peak level over multiple cycles of the repetitive waveform.

20. The apparatus according to claim 19, wherein the processor is configured to identify a pattern of Cheyne-Stokes breathing responsively to a succession of alternating increases and decreases of the peak level.

21. The apparatus according to claim 12, wherein the processor is configured to process the signal so as to detect an occurrence of Cheyne-Stokes breathing.

22. The apparatus according to claim 21, wherein the processor is configured to determine a prognosis of heart failure (HF) in the patient based on the occurrence of the Cheyne-Stokes breathing.

23. A computer-software product, comprising a computer-readable medium in which program instructions are stored, which instructions, when read by a computer, cause the computer to receive a signal indicative of a partial pressure of CO₂ in air expired by a patient during sleep, and to process the signal so as to detect a breathing-related event from a group of events consisting of apneas and hypopneas, and to classify the event as a central event or an obstructive event.

24. The product according to claim 23, wherein the instructions cause the computer to detect a repetitive waveform in the signal prior to the event, and to detect the event by comparing the signal during the event to the detected waveform.

25. The product according to claim 24, wherein the instructions cause the computer to detect an apnea responsively to an interruption of the repetitive waveform.

26. The product according to claim 25, wherein the instructions cause the computer to classify the apnea as central or obstructive responsively to a level of the signal during the interruption.

27. The product according to claim 26, wherein the instructions cause the computer to find peak and baseline values of the repetitive waveform, and to classify the apnea as a central apnea when the level is closer to the peak value than to the baseline value, and as an obstructive apneas when the level is closer to the baseline value than to the peak value.

28. The product according to claim 24, wherein the instructions cause the computer to find shape parameters of the waveform, and to detect a hypopnea responsively to a change in one or more of the shape parameters.

29. The product according to claim 28, wherein the shape parameters comprise a respective slope and a respective duration of one or more phases of the waveform, and wherein the instructions cause the computer to classify the hypopnea as obstructive responsively to a change in the respective slope and the respective duration of at least one of the phases.

30. The product according to claim 24, wherein the instructions cause the computer to find a peak level of the waveform, and to detect a hypopnea responsively to an increase in the peak level over multiple cycles of the repetitive waveform.

31. The product according to claim 30, wherein the instructions cause the computer to identify a pattern of Cheyne-Stokes breathing responsively to a succession of alternating increases and decreases of the peak level.

32. The product according to claim 23, wherein the instructions cause the computer to process the signal so as to detect an occurrence of Cheyne-Stokes breathing.

33. The product according to claim 32, wherein the instructions cause the computer to determine a prognosis of heart failure (HF) in the patient based on the occurrence of the Cheyne-Stokes breathing.
